(19) United States
(12) Patent Application Publication
(10) Pub. No.: US 2002/0095112 A1
(43) Pub. Date: Jul. 18, 2002

(54) Nostril cleaning system

(76) Inventor: Haissam K. Hamdan, Manchester, NH (US)

Correspondence Address: Michael S. Neustel, Suite No. 4, 2534 South University Drive, Fargo, ND, 58103, US

(21) Appl. No.: 09/761,516

(22) Filed: Jan. 16, 2001

(51) Int. Cl. A61M 1/00

(52) U.S. Cl. 604/11; 604/35; 604/36; 604/96.01; 604/104; 604/118

(57) ABSTRACT

A nostril cleaning system for assisting individuals in the efficient cleaning of their nostrils. The nostril cleaning system includes a housing, a control pad having a plurality of control buttons, a plurality of section members pivotally attached within an upper portion of the housing forming a tapered shape, a covering removably positioned about the plurality of section members, and a cover member removably attachable about the upper portion of the housing for enclosing the covering. The covering is preferably comprised of foam material an outer layer and an inner layer wherein the inner layer is denser than the outer layer and capable of absorbing fluids. The control pad is mechanically connected to the section members for controlling their relative positions. When the section members are expanded outwardly, the covering is simultaneously expanded outwardly to engage the inner walls of the nasal passage.

### BACKGROUND OF THE INVENTION

1. Field of the Invention

The present invention relates generally to nostril cleaning devices and more specifically it relates to a nostril cleaning system for assisting individuals in the efficient cleaning of their nostrils.

The external nose consists of bone and cartilage. The hollow internal nose, above the roof of the mouth, is divided by the septum (wall) into two nasal cavities extending from the nostrils to the pharynx. The cavities are lined with a mucous membrane, which is covered with fine hairs that help to filter dust and impurities from the air before it reaches the lungs; the air is also moistened and warmed in its passage. High in each nasal cavity is a small tract of mucous membrane containing olfactory cells. Hair like fibers in these nerve cells, responding to various odors, send impulses along the olfactory nerve to the brain and thus produce the sense of smell. Unfortunately the nasal cavities of an individual often times become coated with a dried layer of mucous and other debris that are irritating to the individual and interferes with the normal functions of the nose. There is therefore a need for a system that effectively cleans the nasal passages of an individual.

2. Description of the Prior Art

Tissue paper and other devices have been in use for years. An individual desiring to clean their nasal passages typically blows nasal mucous and other debris from their nostrils into the tissue paper. Unfortunately, a portion of the mucous and debris often times remains with the nasal passage thereby causing discomfort to the individual.

Examples of patented inventions which are illustrative of such prior art include U.S. Pat. No. 5,584,827 to Korteweg et al; U.S. Pat. No. 1,381,829 to Hartman; U.S. Pat. No. 5,895,408 to Pagan; U.S. Pat. No. 5,800,425 to DeLeonardis; U.S. Pat. No. 5,391,179 to Mezzoli; U.S. Pat. No. 5,531,671 to Bennett; U.S. Pat. No. 2,691,985 to Newsom; U.S. Pat. No. 2,362,704 to McGivern; U.S. Pat. No. 430,934 to Willard; U.S. Pat. No. 4,286,596 to Rubinstein; and U.S. Pat. No. 4,568,326 to Rangaswamy.

While these devices may be suitable for the particular purpose to which they address, they are not as suitable for assisting individuals in the efficient cleaning of their nostrils. Conventional nasal passage cleaning devices do not provide adequate cleaning of the nasal passages of an individual often times leaving a portion of mucous and other debris behind.

In these respects, the nostril cleaning system according to the present invention substantially departs from the conventional concepts and designs of the prior art, and in so doing provides an apparatus primarily developed for the purpose of assisting individuals in the efficient cleaning of their nostrils.

### SUMMARY OF THE INVENTION

In view of the foregoing disadvantages inherent in the known types of nostril cleaning devices now present in the prior art, the present invention provides a new nostril cleaning system construction wherein the same can be utilized for assisting individuals in the efficient cleaning of their nostrils.

The general purpose of the present invention, which will be described subsequently in greater detail, is to provide a new nostril cleaning system that has many of the advantages of the nostril cleaning devices mentioned heretofore and many novel features that result in a new nostril cleaning system which is not anticipated, rendered obvious, suggested, or even implied by any of the prior art nostril cleaning devices, either alone or in any combination thereof.

To attain this, the present invention generally comprises a housing, a control pad having a plurality of control buttons, a plurality of section members pivotally attached within an upper portion of the housing forming a tapered shape, a covering removably positioned about the plurality of section members, and a cover member removably attachable about the upper portion of the housing for enclosing the covering. The covering is preferably comprised of foam material an outer layer and an inner layer wherein the inner layer is denser than the outer layer and capable of absorbing fluids. The control pad is mechanically connected to the section members for controlling their relative positions. When the section members are expanded outwardly, the covering is simultaneously expanded outwardly to engage the inner walls of the nasal passage.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a nostril cleaning system that will overcome the shortcomings of the prior art devices.

A second object is to provide a nostril cleaning system for assisting individuals in the efficient cleaning of their nostrils.

Another object is to provide a nostril cleaning system that is simple and convenient to utilize.

An additional object is to provide a nostril cleaning system that effectively removes hardened mucous from a nasal passage.

A further object is to provide a nostril cleaning system that is capable of fitting within and cleaning various sizes of nasal passages.

Another object is to provide a nostril cleaning system that has a replaceable cleaning head portion.

A further object is to provide a nostril cleaning system that utilizes water in combination with a cleaning head to clean the nasal passage.

Another object is to provide a nostril cleaning system that is reusable.

Another object is to provide a nostril cleaning system that reduces contact between hands and the inner portion of the nose.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is a side view of the present invention.

FIG. 3 is an upper perspective view of the present invention in relation to an individual's nose.

FIG. 4 is an exploded side view of the present invention.

FIG. 5 is a cross sectional view taken along line 5-5 of FIG. 4 illustrating the removable cover.

FIG. 6 is a cross sectional view taken along line 6-6 of FIG. 4 illustrating the removable cover.

FIGS. 7a-c are side views of the present invention being expanded to various widths.

FIGS. 8a-c are side view of the present invention with the cover removed showing the section members being expanded outwardly.

### DESCRIPTION OF THE PREFERRED EMBODIMENT

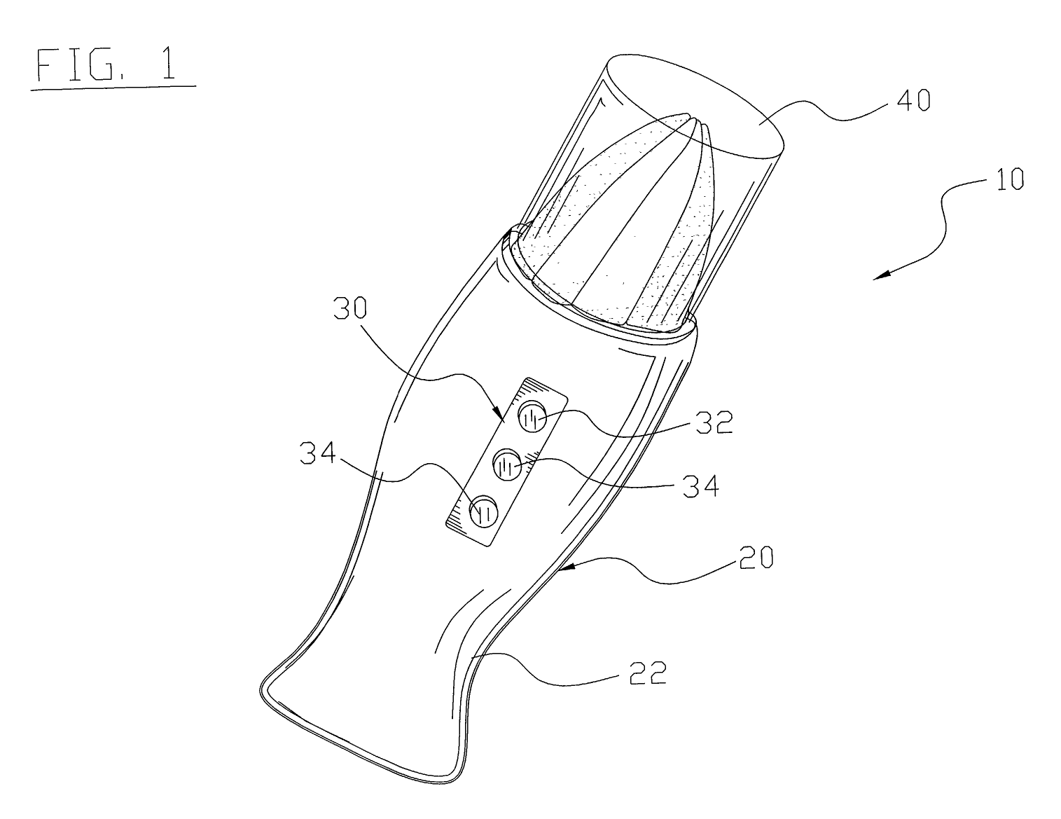

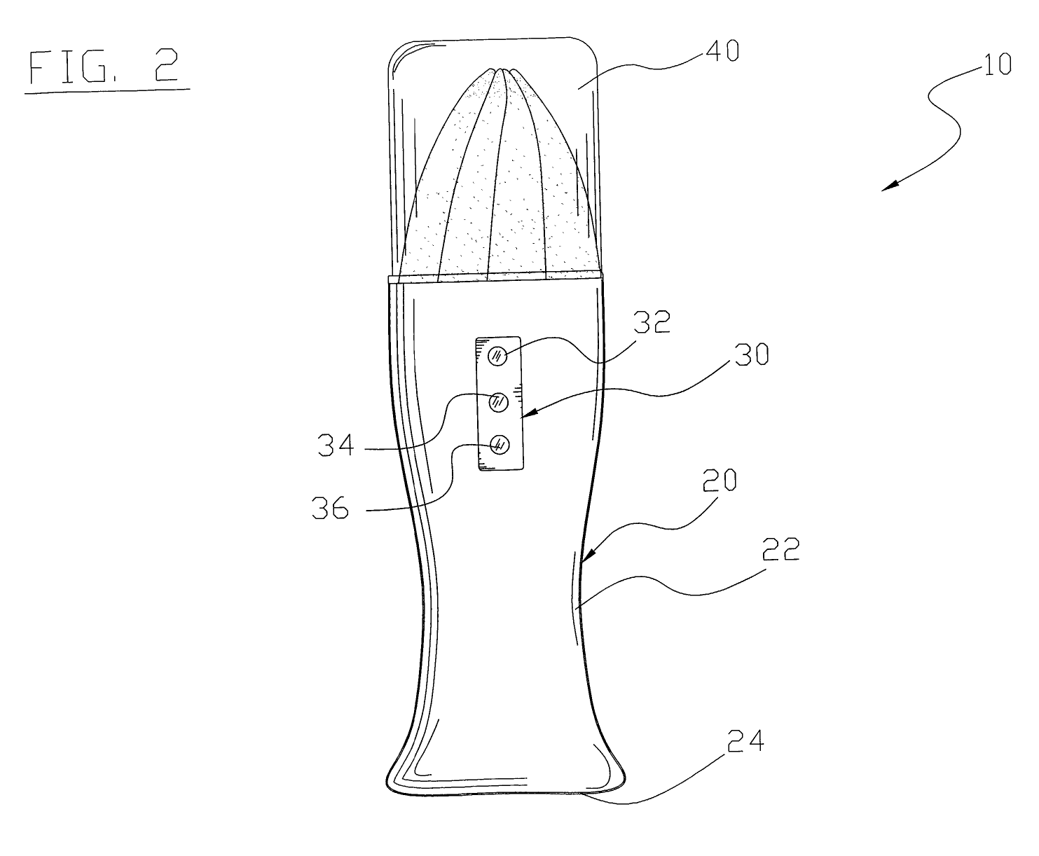

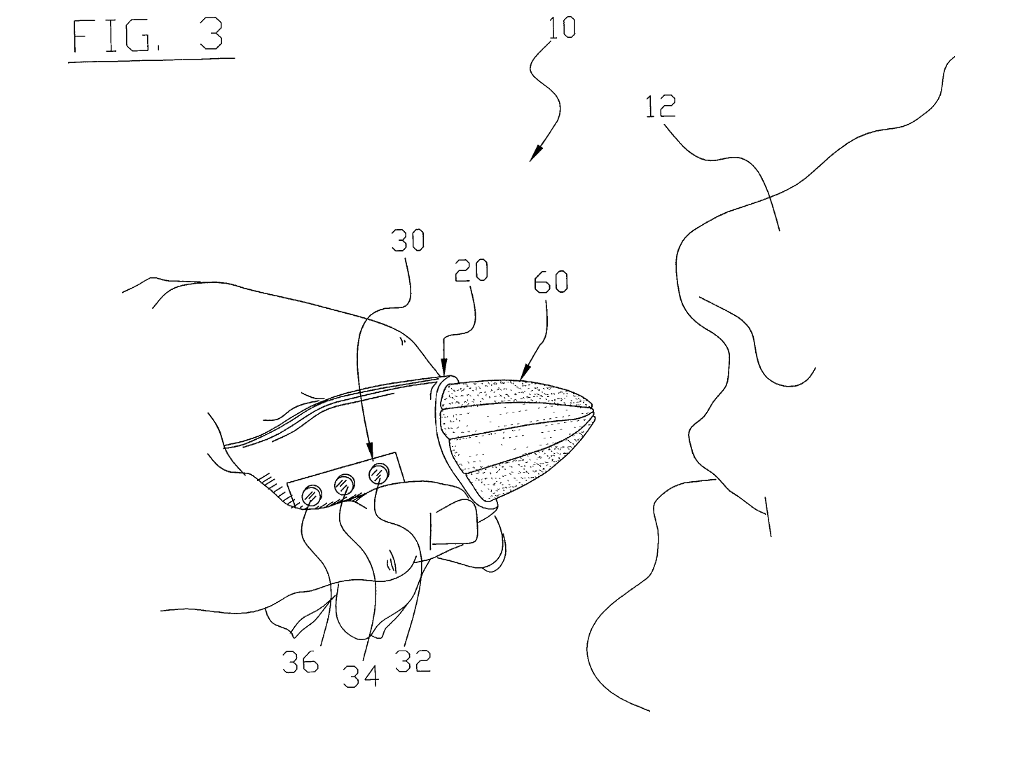

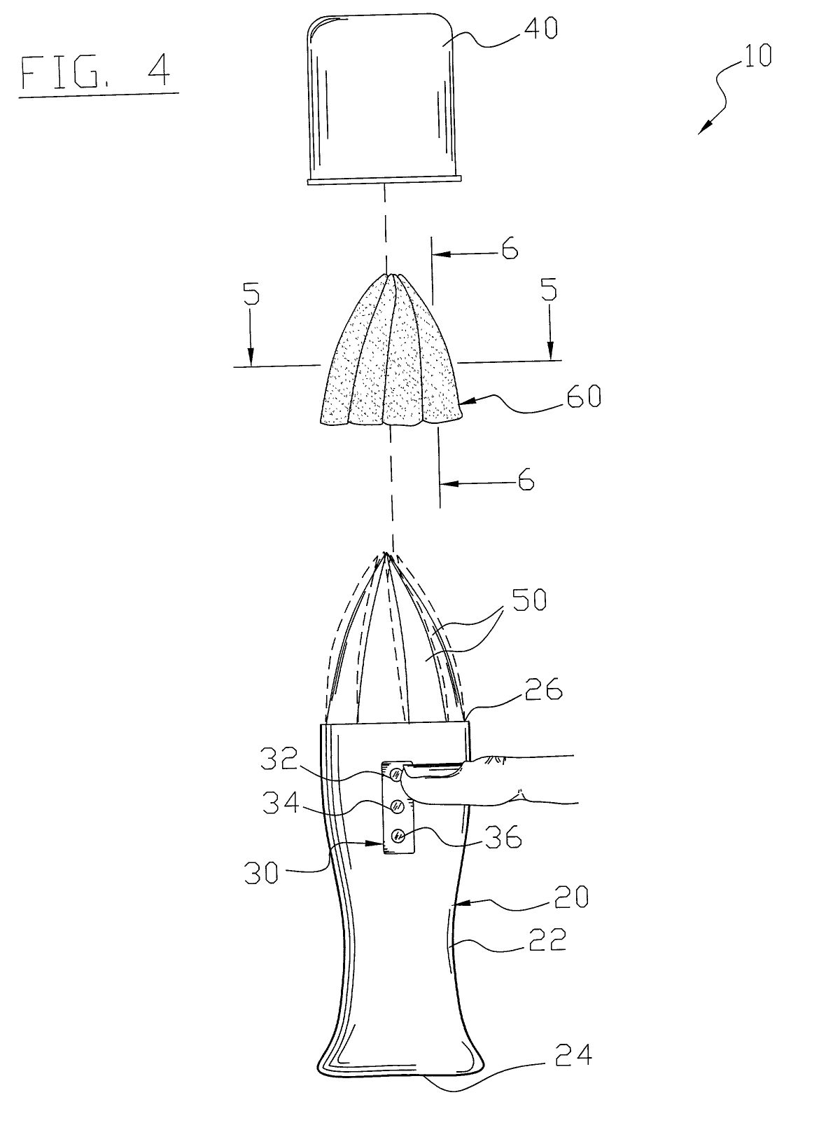

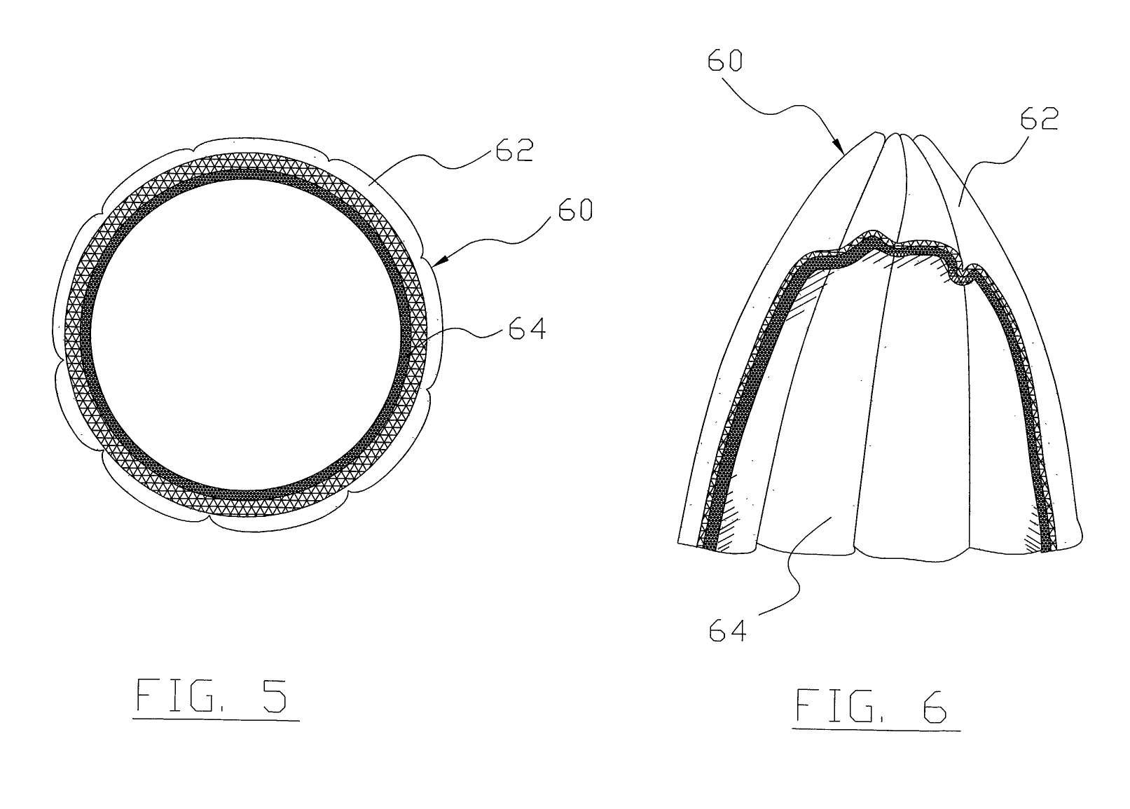

Turning now descriptively to the drawings, in which similar reference characters denote similar elements throughout the several views, FIGS. 1 through 8c illustrate a nostril cleaning system 10, which comprises a housing 20, a control pad 30 having a plurality of control buttons 32, 34, 36, a plurality of section members 50 pivotally attached within an upper portion 26 of the housing 20 forming a tapered shape, a covering 60 removably positioned about the plurality of section members 50, and a cover member 40 removably attachable about the upper portion 26 of the housing 20 for enclosing the covering 60. The covering 60 is preferably comprised of foam material an outer layer 62 and an inner layer 64 wherein the inner layer 64 is denser than the outer layer 62 and capable of absorbing fluids. The control pad 30 is mechanically connected to the section members 50 for controlling their relative positions. When the section members 50 are expanded outwardly, the covering 60 is simultaneously expanded outwardly to engage the inner walls of the nasal passage.

As shown in FIGS. 1, 2, 3, 4, 7a-c and 8a-c of the drawings, the housing 20 is generally comprised of an elongate structure. The housing 20 preferably has a base 24 at the lower end thereof for allowing positioning in an upright position upon various surfaces. The housing 20 further preferably includes a tapered portion 22 for assisting in the gripping by an individual during usage as further shown in the drawings. The housing 20 further includes an upper portion 26 opposite of the base 24 that receives the cover member 40. The housing 20 may be constructed of various structures, designs and materials as can be appreciated by one skilled in the art and the drawings are not illustrative of all possible variations to the housing 20.

The control pad 30 is preferably positioned within the side of the housing 20 for easy access for an individual as shown in FIGS. 1 through 4 of the drawings. The control pad 30 preferably has a plurality of control buttons 32, 34, 36 that provide selective control of the plurality of section members 50. The control buttons 32, 34, 36 may be connected directly to the plurality of section member via a conventional mechanical connection means or indirectly utilizing an electric motor to mechanically move the section members 50 inwardly and outwardly.

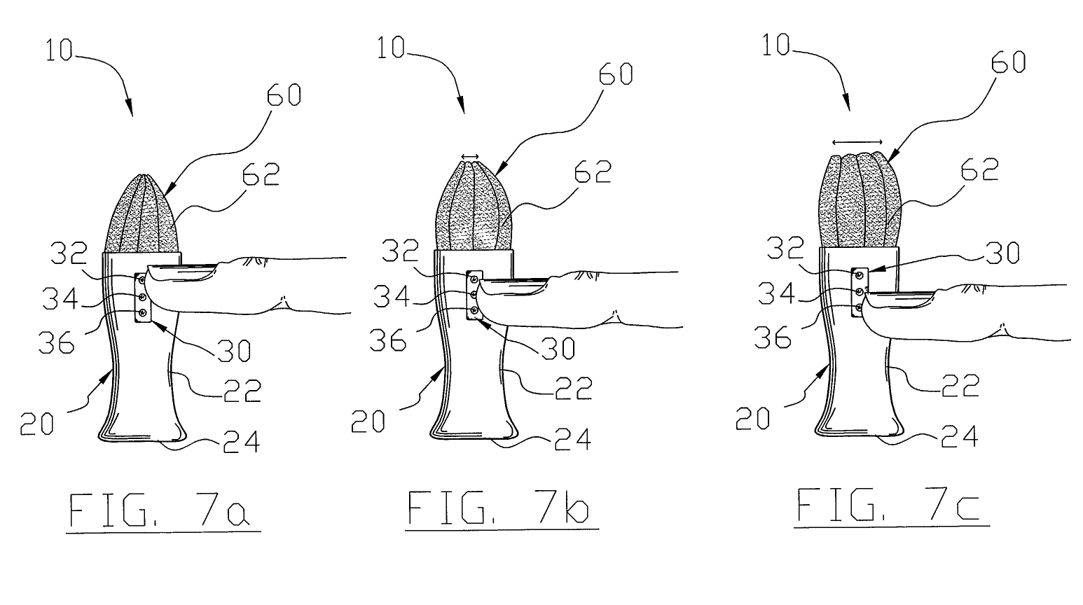

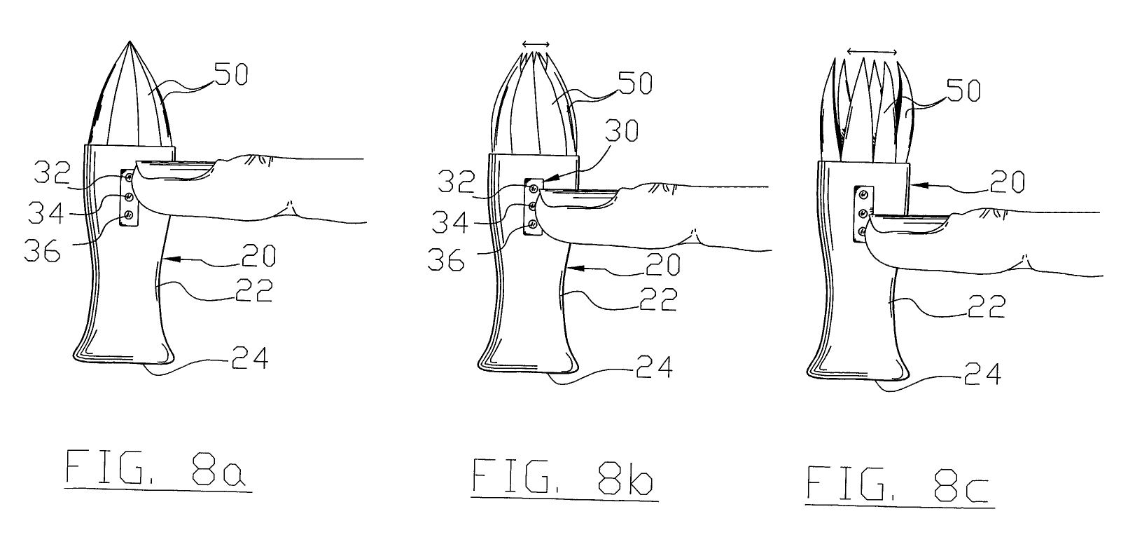

As best shown in FIGS. 7a-c and 8a-c of the drawings, there is preferably a first button 32, a second button 34 and a third button 36 representing a retracted position, a medium extended position, and a fully extended position. When the user depresses the first button 32, the section members 50 are brought inwardly toward one another into the retracted position as shown in FIGS. 7a and 8a of the drawings. When the user depresses the second button 34, the section members 50 are extended outwardly from one another into the medium extended position as shown in FIGS. 7b and 8b of the drawings. When the user depresses the third button 36, the section members 50 are extended outwardly from one another into the fully extended position as shown in FIGS. 7c and 8c of the drawings.

A plurality of section members 50 are pivotally attached to the upper portion 26 of the housing 20 as shown in FIGS. 7a-c and 8a-c of the drawings. Each of the section members 50 is tapered inwardly as best shown in FIG. 8c of the drawings. When the plurality of section members 50 are closed upon one another as shown in FIG. 8a of the drawings they form a relatively solid tapered structure. When the plurality of section members 50 are fully extended as shown in FIG. 8c of the drawings they form a hollow tubular cavity.

As shown in FIGS. 1 through 4 and 7a-c of the drawings, the covering 60 is formed to fit about the plurality of section members 50 and is attached to the upper portion 26 of the housing 20. The covering 60 preferably comprised of a resilient foam material for allowing conforming to the shape of the section members 50 in the various positions as shown in FIGS. 7a-c of the drawings. The covering 60 preferably is comprised of an outer layer 62 and an inner layer 64 wherein the inner layer 64 is denser than the outer layer 62. The covering 60 is preferably fluid absorbent for allowing the absorption of fluids such as medicine or water.

As shown in FIGS. 1, 2 and 4 of the drawings, a cover member 40 is provided to enclose the covering 60 and section members 50 when not in use. The cover member 40 preferably snaps upon the upper portion 26 of the housing 20 similar to conventional snap cover devices commonly utilized.

In use, the user preferably applies a fluid to the covering 60 positioned about the section members 50 to assist in the loosening of hardened mucous and debris within the nasal passage. After the covering 60 has absorbed the desired amount of fluid, the user then positions the covering 60 within one of their nasal passages to be cleansed. After inserting the covering 60 into the nasal passage the desired amount, the user then selects either the second button 34 or the third button 36 depending upon the relative size of their nasal passage. As the user pushes the selected button 34, 36 inwardly, the section members 50 are simultaneously expanded outwardly thereby expanding the size of the covering 60 as shown in FIGS. 7b, 7c, 8b and 8c of the drawings. The user then may rotate and/or reciprocate the nostril cleaning system 10 so that the outer layer 62 of the covering 60 engages and cleanse the interior wall of the nasal passage. During rotation and/or reciprocation of the nostril cleaning system 10, the fluid within the covering 60 is released and engages the mucous and debris within the nasal passage thereby loosening the mucous and debris for removal by engagement of the outer layer 62 of the covering 60. The less dense foam material of the outer layer 62 collects the removed debris from the inner passage of the nose 12. After the user is finished cleaning the nasal passage, the user then positions the covering 60 beneath a steady flow of water or other fluid to cleanse the covering 60 of the collected debris and mucous. If required, the user may replace the covering 60 by simply removing the covering 60 and attaching a new covering 60 about the section members 50.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed to be within the expertise of those skilled in the art, and all equivalent structural variations and relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A nostril cleaning system, comprising:
a housing having an upper portion and a base;
a plurality of section members pivotally attached within said upper portion of said housing;
a covering surrounding said plurality of section members; and
a control pad having a plurality of control buttons positioned within said housing and mechanically connected directly to said plurality of section members for pivoting said section members relative to said housing to a plurality of positions.

2. The nostril cleaning system of claim 1, wherein said plurality of positions includes a retracted position, a medium extended position and a fully extended position.

3. The nostril cleaning system of claim 2, wherein said covering is constructed of a fluid absorbing material.

4. The nostril cleaning system of claim 3, wherein said plurality of section members form a tapered structure when in said retracted position.

5. The nostril cleaning system of claim 4, wherein said plurality of section members expand outwardly from a longitudinal axis of said housing to form a structure broader than said tapered structure.

6. The nostril cleaning system of claim 5, wherein each of said plurality of section members are comprised of an inwardly curved and tapered structure.

7. The nostril cleaning system of claim 6, wherein said plurality of control buttons are comprised of a first button corresponding to said retracted position, a second button corresponding to said medium extended position, and a third button corresponding to said fully extended position.

8. The nostril cleaning system of claim 7, wherein said housing includes a tapered portion.

9. The nostril cleaning system of claim 8, wherein said covering is comprised of an outer layer and an inner layer, wherein said inner layer is denser than said outer layer.

10. The nostril cleaning system of claim 9, wherein said base is formed for allowing said housing to be positioned in an upright position upon a surface.

11. A nostril cleaning system, comprising:
a housing having an upper portion and a base;
a plurality of section members pivotally attached within said upper portion of said housing;
a covering surrounding said plurality of section members;
a motor means mechanically connected to said plurality of section members for pivoting said section members relative to said housing to a plurality of positions; and
a control pad having a plurality of control buttons positioned within said housing and in communication with said motor means for controlling the operation of said motor means.

12. The nostril cleaning system of claim 11, wherein said plurality of positions includes a retracted position, a medium extended position and a fully extended position.

13. The nostril cleaning system of claim 12, wherein said covering is constructed of a fluid absorbing material.

14. The nostril cleaning system of claim 13, wherein said plurality of section members form a tapered structure when in said retracted position.

15. The nostril cleaning system of claim 14, wherein said plurality of section members expand outwardly from a longitudinal axis of said housing to form a structure broader than said tapered structure.

16. The nostril cleaning system of claim 15, wherein each of said plurality of section members are comprised of an inwardly curved and tapered structure.

17. The nostril cleaning system of claim 16, wherein said plurality of control buttons are comprised of a first button corresponding to said retracted position, a second button corresponding to said medium extended position, and a third button corresponding to said fully extended position.

18. The nostril cleaning system of claim 17, wherein said housing includes a tapered portion.

19. The nostril cleaning system of claim 18, wherein said covering is comprised of an outer layer and an inner layer, wherein said inner layer is denser than said outer layer.

20. The nostril cleaning system of claim 19, wherein said base is formed for allowing said housing to be positioned in an upright position upon a surface.
